(19) United States
(12) Patent Application Publication
(10) Pub. No.: US 2020/0268243 A1
(43) Pub. Date: Aug. 27, 2020

(54) Cannula and Obturator with a Transparent Tip with an Opaque Component

(71) Applicant: Rebound Therapeutics Corporation, Irvine, CA (US)

(72) Inventors: Peter G. Davis, Irvine, CA (US); Ross Tsukashima, Irvine, CA (US); Donald Joseph Fuller, Irvine, CA (US)

(73) Assignee: Rebound Therapeutics Corporation, Irvine, CA (US)

(21) Appl. No.: 16/793,690

(22) Filed: Feb. 18, 2020

Related U.S. Application Data
(60) Provisional application No. 62/809,445, filed on Feb. 22, 2019.

(51) Int. Cl. A61B 1/313; A61B 17/34; A61B 34/20; A61B 1/00

(52) U.S. Cl. CPC A61B 1/313; A61B 17/3421; A61B 34/20; A61B 2017/00907; A61B 1/00096; A61B 2017/3425; A61B 17/3496

(57) ABSTRACT

A cannula and obturator system with an obturator having a narrow shaft and a larger distal tip, which is transmissive to visible light (transparent or translucent). The obturator tip has a pointed, acutely conical distal tip, and includes an optically opaque component, such as a rod, disposed along the central axis of the obturator tip, which serves to prevent image reversal.

This application claims priority to U.S. Provisional Application 62/809,445, filed Feb. 22, 2019, which is pending.

### FIELD OF THE INVENTIONS

The inventions described below relate to the field of minimally invasive surgery.

### BACKGROUND OF THE INVENTIONS

In our previous U.S. patent application, we disclose a cannula and camera system with an obturator comprising a small diameter shaft and larger diameter, transparent obturator tip that obturates the cannula at its distal end. Using the system, a surgeon can advance the cannula into the brain while viewing tissue distal to the obturator tip, through the obturator tip. The obturator tip may reverse the image of the tissue distal to the obturator tip, presenting at its proximal surface an image of tissue distal to the tip (at its distal surface) which is reversed, depending on its construction.

### SUMMARY

The devices and methods described below provide for improved visualization of the brain during minimally invasive surgery. The device comprises a cannula system with a camera mounted on the proximal end of the cannula with a view into the cannula lumen and the tissue within and below the lumen, and a obturator comprising a narrow shaft and a larger distal tip, which is transmissive to visible light (transparent or translucent), so that the tissue distal to the tip is at least partially visible, through the tip, from the proximal end of the cannula. The obturator tip preferably has a pointed, acutely conical distal tip, and includes an optically opaque component, such as a rod, disposed along the central axis of the obturator tip, which serves to prevent image reversal.

The small cross-section obturator shaft is much smaller than the inner diameter of the cannula, affording a sizable annular or circular space between the shaft and the cannula wall to provide good visibility (from the camera) of the proximal surface of the obturator tip. Lights, if necessary, may be provided in the cannula to illuminate the distal end of the obturator tip and cannula or tissue near the distal end of the cannula (lighting may instead be provided from a source outside the assembly, or from lights mounted on the proximal end of the cannula or any combination of the foregoing). Light reflected by tissue near the distal surface of the obturator tip passes through the obturator and out of the proximal surface of the obturator tip, so that a surgeon inserting or manipulating the assembly can easily see that the obturator tip is near brain tissue (which is white to gray) or blood (which is red to black).

### BRIEF DESCRIPTION OF THE DRAWINGS

FIG. 1 illustrates the head of a patient with an area requiring surgical intervention.

FIGS. 2 through 5 illustrate the cannula and obturator, in which the obturator is provided with an opaque component within a transparent tip.

### DETAILED DESCRIPTION OF THE INVENTIONS

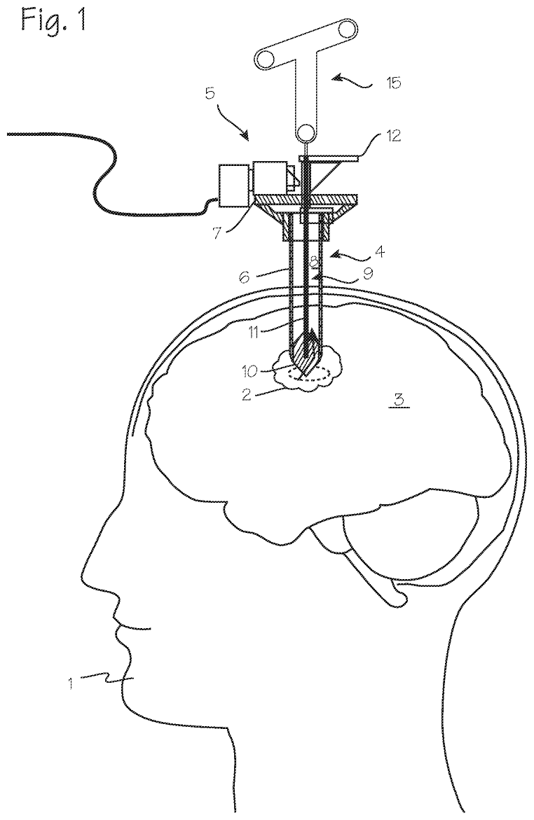

FIG. 1 illustrates a patient 1 with a blood mass 2 in the brain 3 that necessitates surgical intervention. A cannula 4 has been inserted into the brain, with the distal end of the cannula proximate the blood mass. (Though illustrated in the context of brain surgery, the system can be used in any surgery.) A camera 5 is mounted on the proximal rim of the cannula, with a portion of the camera overhanging the rim of the cannula and disposed over the lumen of the cannula, and is operable to obtain video or still images of the blood mass or other tissue at the distal end of the cannula. The cannula comprises a cannula tube 6, with a distal end 6d adapted for insertion into the body of the patient, and the proximal end 6p which remains outside the body during use. The camera 5 is mounted on the proximal end 6p of the cannula tube via a mounting structure 7 secured to the proximal end of the cannula. The camera, which may include or be fitted with a prism, a reflector or other mirror structure or optical element, overhangs the lumen 8 of the cannula tube. If the camera is small compared to the cannula lumen, the camera may be used without the prism or reflector, and may be oriented with its viewing axis aligned along the long axis of the cannula.

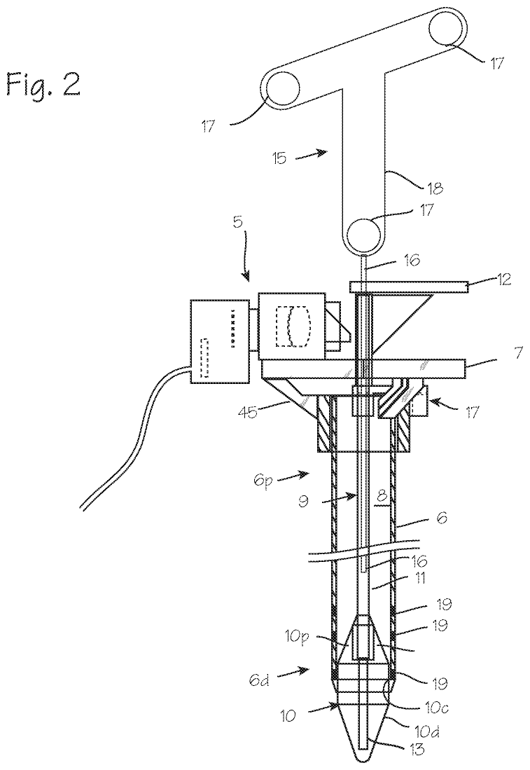

FIGS. 1 and 2 also illustrate the obturator 9. The obturator comprises the obturator tip 10, shaft 11 and a handle 12. The obturator tip is preferably a solid structure with a conically convex distal surface 10d, a conically convex proximal surface 10p, and an axially short circumferential surface 10c. The tip, in the region of the circumferential surface, has an outer diameter (a transverse diameter, along a plane perpendicular to the long axis of the cannula, and corresponding to the transverse cross-sectional diameter of the cannula) that closely matches the inner diameter of the cannula, but allows easy longitudinal translation of the tip through the lumen of the cannula. The tip, configured as shown in FIG. 1, will act as a lens, such that light rays (represented the arrows) are refracted through the tip, and bent such that any “image” passing through the tip, when formed as illustrated, may be reversed. The tip may have a rectilinear longitudinal cross-section, with a central cylindrical portion and distal and proximal conical portions, as illustrated, or a more rounded cross-section. The distal taper preferably ends in an acutely pointed tip which is preferred for use in the brain. As illustrated, the preferred conical tip has an apex angle of about 35° to 45°. The sharpened distal tip extending distally of the cannula tube facilitates advancement of the assembly through brain tissue. The distal surface and proximal surface need not be symmetric about the longitudinal axis, or symmetric about a transverse axis. For example, the distal surface may be pointed, with a rectilinear cross-section, while the proximal surface is pointed, rounded, or flat. Though not preferred for use in the brain, the distal surface of the obturator tip may be blunt, such as spherical or spheroid, and the entire obturator tip may be formed as a sphere, a spheroid, a prolate spheroid (a football, rugby ball), and oblate spheroid, or an ovoid (egg-shaped).

The obturator tip is optically transmissive, not optically opaque, and may be optically transparent or optically translucent. The transmittance of the tip need only be adequate, in the visible spectrum, to pass the color of tissue in contact with the distal surface, given the brightness of any illumination provided by the light sources, to provide enough transmitted light to the camera and/or eye of the surgeon to allow the color of tissue around the distal tip to be discerned from light transmitted through the proximal surface of the tip. The obturator tip has a transverse cross-section closely matching a transverse cross-section of the cannula, and is slidable within the cannula tube, and positionable within the cannula tube such that the proximal end of the obturator shaft extends proximally out of the cannula proximal end while the tapered distal tip extends out of the cannula distal end. The obturator shaft has a transverse cross-section smaller than the lumen of the cannula and smaller that the transverse cross-section of the obturator tip, so that the proximal surface of the obturator tip is visible from the proximal end of the cannula, through the lumen, when the obturator tip is disposed within the cannula such that the tapered distal tip extends out of the cannula distal end.

The tip may be made of glass, silica, acrylic, polycarbonate, silicone, nylon, polyamides or copolymers or any other material suitable for use in a medical device. The obturator tip surface may be polished or frosted. The obturator tip may optionally comprise radiopaque substances (elements or compounds such as platinum particles, for example) to render the tip radiopaque, so that it appears distinctly under fluoroscopy during surgery. The obturator tip may optionally comprise sensors such as pH sensors, impedance sensors, force sensors, glucose sensors, etc., to assist in detecting a blood mass or CSF and distinguishing them from surrounding brain tissue.

The proximal surface of the tip, which tapers to a small diameter in the proximal direction, also provides for clearance of the tip when the obturator must be removed to make room for other devices, despite the position of the camera assembly at the distal end of the cannula tube.

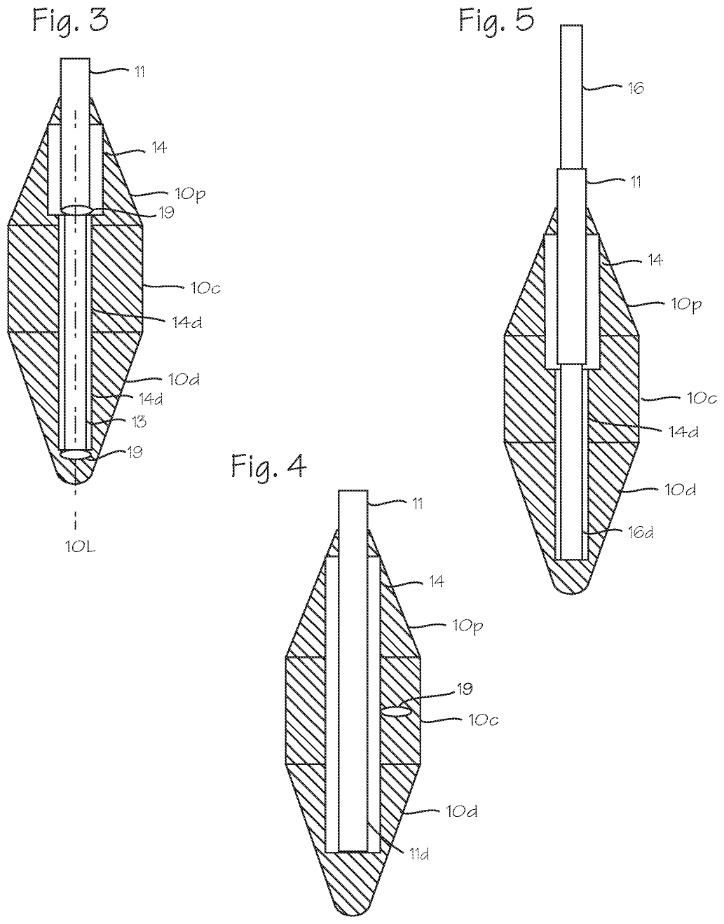

As shown in FIGS. 2 through 5, the obturator tip is modified by the inclusion of an opaque component or components, such as a rod 13 or any opaque substance which serves to prevent image reversal, disposed along the central axis of the obturator tip. The opaque component extends through the central cylindrical portion defined by the circumferential surface 10c and much of the distal tip of the obturator tip (that is, the distal conical portion or the portion bounded by the conically convex distal surface 10d). The opaque component is disposed along the central long axis 10L of the obturator tip, and is preferably coaxial with the obturator shaft, leaving the periphery of the obturator tip clear for maximum passage of light, and is preferably a single discrete component or single compact mass of substance. Due to the presence of this opaque component, the lensing effect of the obturator tip will be disrupted, and images of tissue on the left side of the distal tip will appear to the surgeon on the left side of the proximal surface, and images of tissue on the right side of the distal tip will appear to the surgeon on the right side of the proximal surface, etc. Also shown in FIGS. 2 and 3, the proximal conical section of the obturator tip includes a bore 14 which accommodates the distal end of the obturator shaft. This bore can terminate distally within the proximal conical section, or within the central cylindrical section, or even within the distal conical section. The opaque component 13 is disposed within a bore as well, and this additional bore 14d may be of smaller diameter than the bore that accommodates the shaft 11, or it may be the same diameter (and may be formed as a continuation of the shaft bore 14 or may be formed from the distal surface such that it is blind to the shaft bore). The opaque component may be provided as a component 13 separate from the obturator shaft 11, as shown in FIG. 3, with a transverse dimension (a radial diameter, for example, if the radial cross section is circular) which is smaller than the transverse dimension of the obturator shaft 11. In embodiments in which the bore which accommodates the obturator shaft terminates in the distal conical section, the obturator shaft distal end 16d may terminate also in the distal conical section, and the distal tip of the obturator shaft may serve as the optically opaque component 13, such that the opaque component is not a component separate from the obturator shaft 11. This is illustrated in FIG. 4, in which the obturator tip comprises a proximal portion 10p and a central cylindrical portion 10c, and the tapered distal tip 10d is a distal conical portion, and the obturator further comprises a bore 14, 14d extending from a proximal-most extent of the proximal portion, distally into the central cylindrical portion, and in which the distal end 10d of the shaft is disposed within the bore, where the bore has a length such that, when proximal end of the obturator shaft extends proximally out of the cannula proximal end the tapered distal tip extends out of the cannula distal end, the bore 14, 14d terminates distally at a point distal to the distal end of the cannula tube. In this embodiment, the opaque component may comprise the distal end 11d of the shaft. The opaque component/distal end of the obturator shaft may have a transverse dimension which is smaller than the transverse dimension of the obturator shaft 11, or, as shown, it may have the same transverse dimension as the obturator shaft.

In embodiments in which the shaft bore terminates in the proximal conical section, or central cylindrical section, the neuro-navigation stylet distal tip 16d may extend distally beyond the shaft bore 14 (the larger bore which accommodated the shaft 11), and extend into the distal conical section (in the smaller bore 14d extending beyond the shaft bore, of smaller diameter that the shaft bore), for example into a smaller diameter bore the extends distally from the shaft bore, to serve as the opaque component. This is shown in FIG. 5, in which the distal end of the navigation stylet extends well past the open distal end of the cannula tube, and into the distal conical section at the tip of the obturator which, when assembled with the cannula tube, extends past the open distal end of the cannula tube. In this embodiment, the neuro-navigation stylet has a distal end a proximal end, and is sized and dimensioned for insertion in a lumen of the obturator shaft, such that when proximal end of the obturator shaft extends proximally out of the cannula proximal end the tapered distal tip extends out of the cannula distal end, and the neuro-navigation stylet is disposed within the lumen of the obturator shaft, the distal end of the neuro-navigation stylet terminates distally at a point distal to the distal end of the cannula tube. The distal end of the neuro-navigation stylet may serve as the opaque component comprises.

The shaft 11 may be a solid rod or a tube, with a small diameter, or transverse cross-section, compared to the cannula lumen and the obturator tip, so that the tip proximal surface can be viewed from the cannula proximal end. If provided as a tube, the lumen of the shaft may accommodate a neuro-navigation stylet or probe 15 with markers detectable by the neuro-navigation system, useful for guidance of the assembly into the brain. The rod 16 of the neuro-navigation stylet may be inserted into the lumen of tubular shaft, as shown, so that the assembled cannula, obturator and stylet may be tracked by a neuro-navigation system, through tracking of the markers 17 on a frame 18 to aid in accurate placement of the distal tip of the assembly. The shaft 11 may also accommodate a neuro starburst connection. In embodiments which include a neuro-navigation stylet, the distal tip of the stylet can terminate at any point within the obturator shaft, and may terminate proximal to the obturator tip, or within the obturator tip at a point proximal to the distal edge of the cannula or distal to the distal edge of the cannula.

In use, a surgeon will assemble the cannula tube, obturator, and, optionally, the neuro-navigation stylet for insertion into the body of a patient to gain access to a surgical workspace. For example, the method may entail providing a cannula system comprising the cannula tube, the obturator and the neuro-navigation stylet and assembling the cannula tube, obturator and neuro-navigation stylet such that the obturator is disposed within the cannula tube with the obturator tip extending distally from the cannula distal end and the distal end of end the neuro-navigation stylet extends distally from the obturator shaft and into the distal tip of the obturator tip to provide the opaque component within the distal tip, and advancing the assembled cannula system into the body while observing body tissue distal to the cannula tube, through the obturator tip, from the proximal end of the cannula tube. Where a neuro-navigation stylet is not to be used as the opaque component, the method may entail providing a cannula system comprising the cannula tube and the obturator, where the obturator tip further includes an opaque component disposed in the distal tip, and assembling the cannula tube and obturator such that the obturator is disposed within the cannula tube with the obturator tip extending distally from the cannula distal end such that the opaque component within the distal tip is disposed distally of the distal end of the cannula tube, and advancing the assembled cannula system into the body while observing body tissue distal to the cannula tube, through the obturator tip, from the proximal end of the cannula tube.

To aid in visualization, lights may be incorporated into the obturator tip to cast light on tissue distal to the tip and make it easier to see the tissue through the obturator tip from the proximal end of the cannula tube. As shown in FIG. 3, a light such as an LED 19, may be disposed within the distal conical portion, and may be embedded in the material of the obturator or fixed at the bottom of the bore 14d which accommodates the opaque component. An LED may instead be disposed within the proximal portion (FIG. 3) or the cylindrical portion (FIG. 4), and may be embedded in the material of the obturator or fixed at the bottom of the bore 14 which accommodates the obturator shaft 11. The light source may be employed in embodiments which omit the opaque component. Wiring necessary to power the light can be disposed within or about the obturator shaft.

While the preferred embodiments of the devices and methods have been described in reference to the environment in which they were developed, they are merely illustrative of the principles of the inventions. The elements of the various embodiments may be incorporated into each of the other species to obtain the benefits of those elements in combination with such other species, and the various beneficial features may be employed in embodiments alone or in combination with each other. Other embodiments and configurations may be devised without departing from the spirit of the inventions and the scope of the appended claims.

We claim:

1. A cannula system for accessing a blood mass in the brain of a patient, said cannula system comprising:
a cannula comprising a cannula tube with a proximal end and a distal end and a lumen extending from the proximal end to the distal end; and
an obturator comprising an obturator shaft having a proximal end and a distal end, and an obturator tip disposed on the distal end of said obturator shaft, said obturator tip being optically transmissive, said obturator tip having a transverse cross-section closely matching a transverse cross-section of the cannula, said obturator tip having a tapered distal tip, said obturator being slidable within the cannula tube, and positionable within the cannula tube such that the proximal end of the obturator shaft extends proximally out of the cannula proximal end while the tapered distal tip extends out of the cannula distal end, wherein the obturator shaft has a transverse cross-section smaller than the lumen of the cannula and smaller that the transverse cross-section of the obturator tip, whereby the proximal surface of the obturator tip is visible from the proximal end of the cannula, through the lumen, when the obturator tip is disposed within the cannula such that the tapered distal tip extends out of the cannula distal end; and
an optically opaque component disposed along a central longitudinal axis of the obturator tip, within the tapered distal tip.

2. The cannula system of claim 1, wherein:
the obturator tip comprises a proximal portion, a central cylindrical portion, and wherein the tapered distal tip is a distal conical portion, and the obturator further comprises a bore extending from a proximal-most extent of the proximal portion, distally into the central cylindrical portion, and the distal end of the shaft is disposed within the bore, where the bore has a length such that, when proximal end of the obturator shaft extends proximally out of the cannula proximal end the tapered distal tip extends out of the cannula distal end, the bore terminates distally at a point distal to the distal end of the cannula tube.

3. The cannula system of claim 2, wherein:
the shaft extends distally into the distal conical portion of the obturator tip, and the opaque component comprises the distal end of the shaft.

4. The cannula system of claim 2, wherein:
the shaft terminates distally in the proximal portion of the obturator tip, and the opaque component a separate component separate from the shaft.

5. The cannula system of claim 1, further comprising:
a neuro-navigation stylet having a distal end a proximal end, said neuro-navigation stylet sized and dimensioned for insertion in a lumen of the obturator shaft; wherein
when proximal end of the obturator shaft extends proximally out of the cannula proximal end the tapered distal tip extends out of the cannula distal end, and the neuro-navigation stylet is disposed within the lumen of the obturator shaft, the distal end of the neuro-navigation stylet terminates distally at a point distal to the distal end of the cannula tube.

6. The cannula system of claim 5, wherein:
the neuro-navigation stylet terminates distally within the distal conical portion of the obturator tip, and the opaque component comprises the distal end of the neuro-navigation stylet.

7. The cannula system of claim 2, wherein:
the distal conical portion has an apex angle in the range of 35 to 45 degrees.

8. The cannula system of claim 2, wherein:
the distal conical portion comprises a sharpened distal tip extending distally of the cannula tube to facilitate advancement of the assembly through brain tissue.

9. A cannula system for accessing a blood mass in the brain of a patient, said cannula system comprising:
a cannula comprising a cannula tube with a proximal end and a distal end and a lumen extending from the proximal end to the distal end; and
an obturator comprising an obturator shaft having a proximal end and a distal end, and an obturator tip disposed on the distal end of said obturator shaft, said obturator tip being optically transmissive, said obturator tip having a transverse cross-section closely matching a transverse cross-section of the cannula, said obturator tip having a distal tip, said obturator being slidable within the cannula tube, and positionable within the cannula tube such that the proximal end of the obturator shaft extends proximally out of the cannula proximal end while the tapered distal tip extends out of the cannula distal end, wherein the obturator shaft has a transverse cross-section smaller than the lumen of the cannula and smaller that the transverse cross-section of the obturator tip, whereby the proximal surface of the obturator tip is visible from the proximal end of the cannula, through the lumen, when the obturator tip is disposed within the cannula such that the tapered distal tip extends out of the cannula distal end; and
a light disposed within the obturator tip.

10. A method of inserting a cannula into the body of a patient to access a surgical workspace within the body of the patient, said method comprising the steps of:
providing a cannula system comprising:
a cannula comprising a cannula tube with a proximal end and a distal end and a lumen extending from the proximal end to the distal end; and
an obturator comprising an obturator shaft having a proximal end and a distal end, and an obturator tip disposed on the distal end of said obturator shaft, said obturator tip being optically transmissive, said obturator tip having a transverse cross-section closely matching a transverse cross-section of the cannula, said obturator tip having a distal tip, said obturator being slidable within the cannula tube, and positionable within the cannula tube such that the proximal end of the obturator shaft extends proximally out of the cannula proximal end while the distal tip extends out of the cannula distal end, wherein the obturator shaft has a transverse cross-section smaller than the lumen of the cannula and smaller that the transverse cross-section of the obturator tip, whereby the proximal surface of the obturator tip is visible from the proximal end of the cannula, through the lumen, when the obturator tip is disposed within the cannula such that the distal tip extends out of the cannula distal end; wherein
the obturator tip further comprises an opaque component disposed in the distal tip; and
assembling the cannula tube and obturator such that the obturator is disposed within the cannula tube with the obturator tip extending distally from the cannula distal end such that the opaque component within the distal tip is disposed distally of the distal end of the cannula tube; and
advancing the assembled cannula system into the body while observing body tissue distal to the cannula tube, through the obturator tip, from the proximal end of the cannula tube.
